(19) United States
(12) Patent Application Publication
(10) Pub. No.: US 2023/0310194 A1
(43) Pub. Date: Oct. 5, 2023

(54) KNEE BRACE AND PROCESS OF MAKING SAME

(71) Applicant: Hung-Yu HSIEH, Changhua County (TW)

(72) Inventor: Hung-Yu HSIEH, Changhua County (TW)

(21) Appl. No.: 17/693,684

(22) Filed: Mar. 14, 2022

(51) Int. Cl. A61F 5/01

(52) U.S. Cl. CPC A61F 5/0109; A61F 5/0118

(57) ABSTRACT

In one embodiment a knee brace includes a tubular cushioning body; two sheaths disposed at two opposite sides of the tubular cushioning body respectively; and two sets of a plurality of elastic strips inserted into the sheaths respectively. There are further provided two tubes with the sets of the plurality of elastic strips inserted thereinto respectively. The tubes are inserted into the sheaths respectively.

### FIELD OF THE INVENTION

The invention relates to pads and more particularly to a knee brace and process of making same having improved characteristics.

### BACKGROUND OF THE INVENTION

Conventionally, knee braces have been used in athletic and orthopedic applications to protect a wearer’s knee from impact. A typical tubular shaped knee brace A is shown in FIGS. 1 and 2 and comprises a plurality of elastic strips B at either side. The knee brace A is worn on the knee to shield the knee against impact. The leg C may be bent by pivoting about the knee as indicated by two solid arrows in exercise. However, each of the elastic strips B may exert a force (as indicated by two arrows in broken lines) to oppose the bending force. Unfortunately, the counterforce compromises the desired knee padding effect of the knee brace A.

Thus, the need for improvement still exists.

### SUMMARY OF THE INVENTION

It is therefore one object of the invention to provide a knee brace comprising a tubular cushioning body; two sheaths disposed at two opposite sides of the tubular cushioning body respectively; and two sets of a plurality of elastic strips inserted into the sheaths respectively.

The above and other objects, features and advantages of the invention will become apparent from the following detailed description taken with the accompanying drawings

### BRIEF DESCRIPTION OF THE DRAWINGS

FIG. 1 is a perspective view of a conventional knee brace;

FIG. 2 is an environmental view showing the conventional knee brace worn on the knee of a person;

FIG. 3 illustrates a first step of a process of making a knee brace according to the invention;

FIG. 4 illustrates a second step of the process of making a knee brace according to the invention;

FIG. 5 illustrates a third step of the process of making a knee brace according to the invention;

FIG. 6 illustrates a fourth step of the process of making a knee brace according to the invention;

FIG. 7 illustrates a fifth step of the process of making a knee brace according to the invention;

FIG. 8 illustrates a sixth step of the process of making a knee brace according to the invention;

FIG. 9A is a perspective view of a knee brace according to the invention;

FIG. 9B is a side elevation schematically showing the knee brace of FIG. 9A;

FIG. 10 is a perspective view of the knee brace being expanded horizontally;

FIG. 11 is an environmental view showing the knee brace worn on the knee of a person;

FIG. 12 is an environmental view showing the knee brace on the knee which bends and straighten in exercise in a first configuration of the invention;

FIG. 13 an environmental view showing the elbow brace worn on the elbow of the person in a second configuration of the invention;

FIG. 14 an environmental view showing the knee brace put on a joining portion of the hand and the shoulder by putting a looped strap over the shoulder in a third configuration of the invention;

FIG. 15 is a side view of the shoulder brace of FIG. 14; and

FIG. 16 illustrates a sixth step of the process of making a knee brace according to another embodiment of the invention.

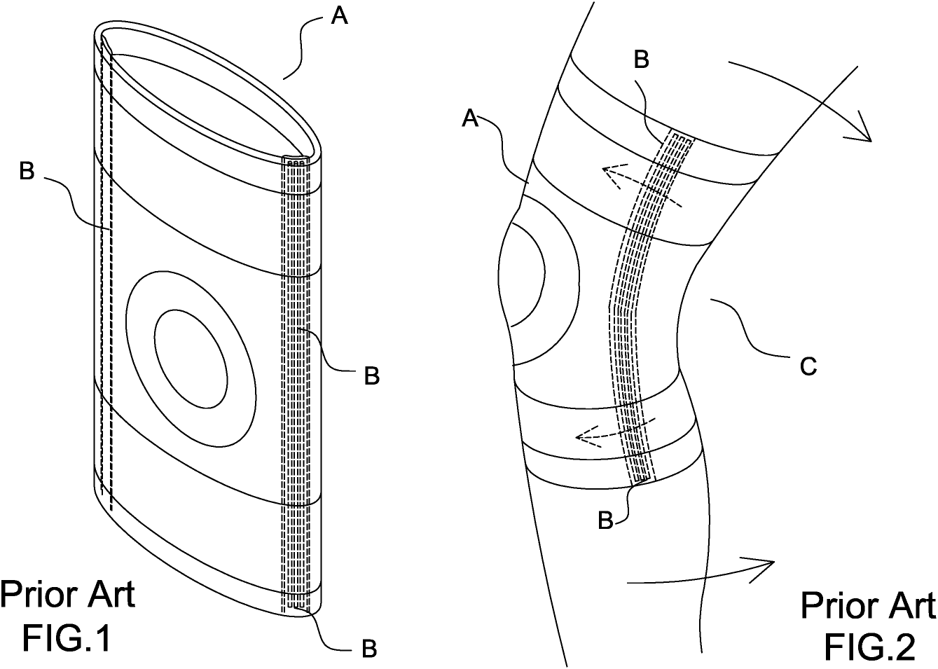

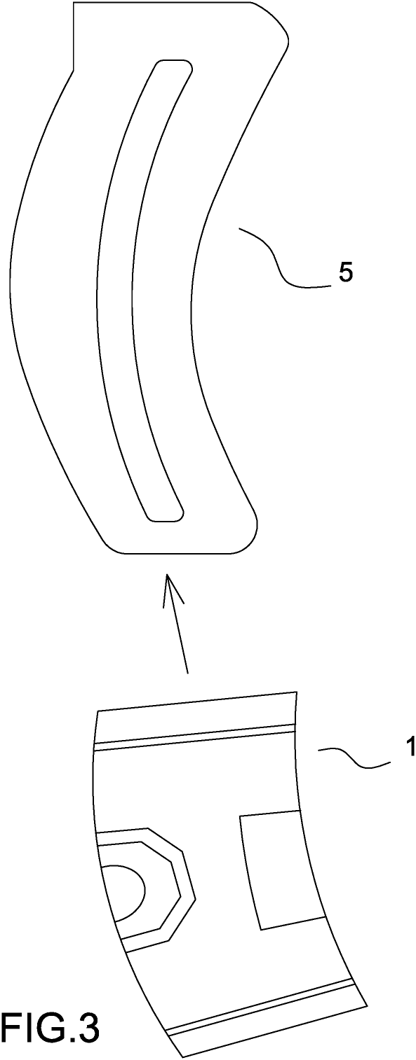

### DETAILED DESCRIPTION OF THE INVENTION

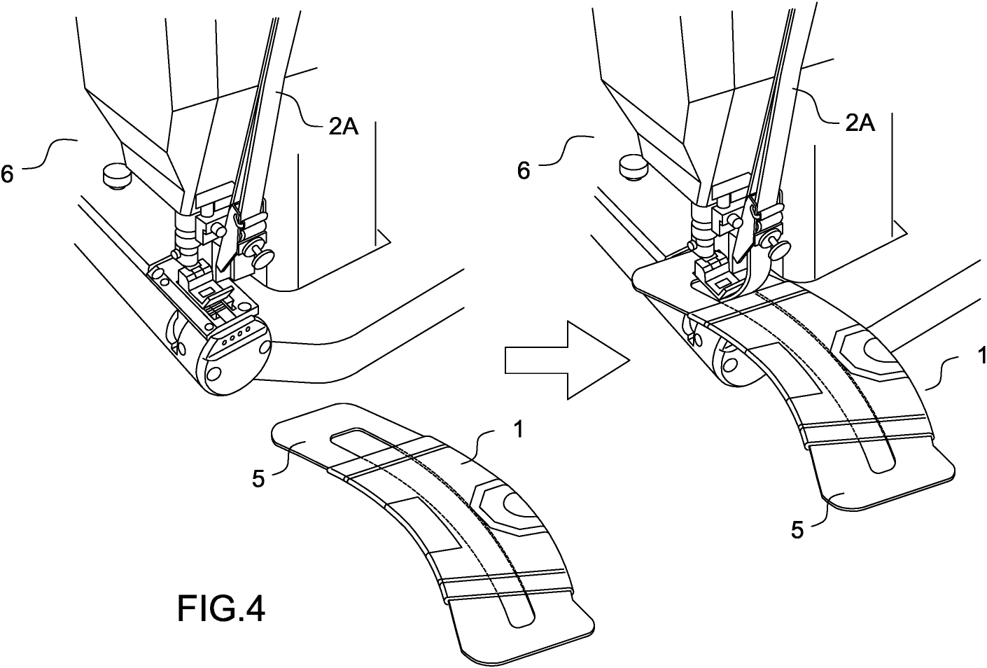

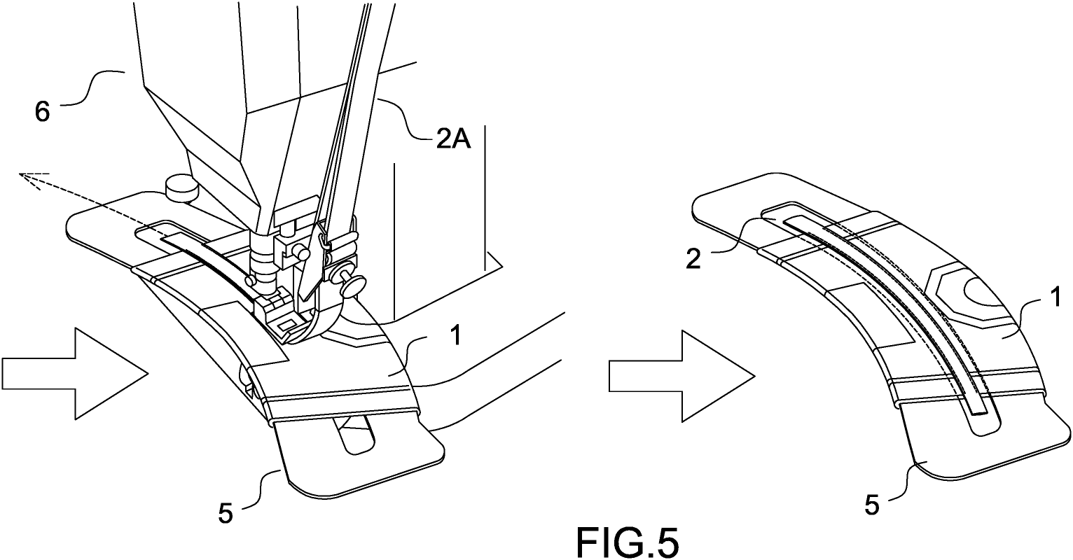

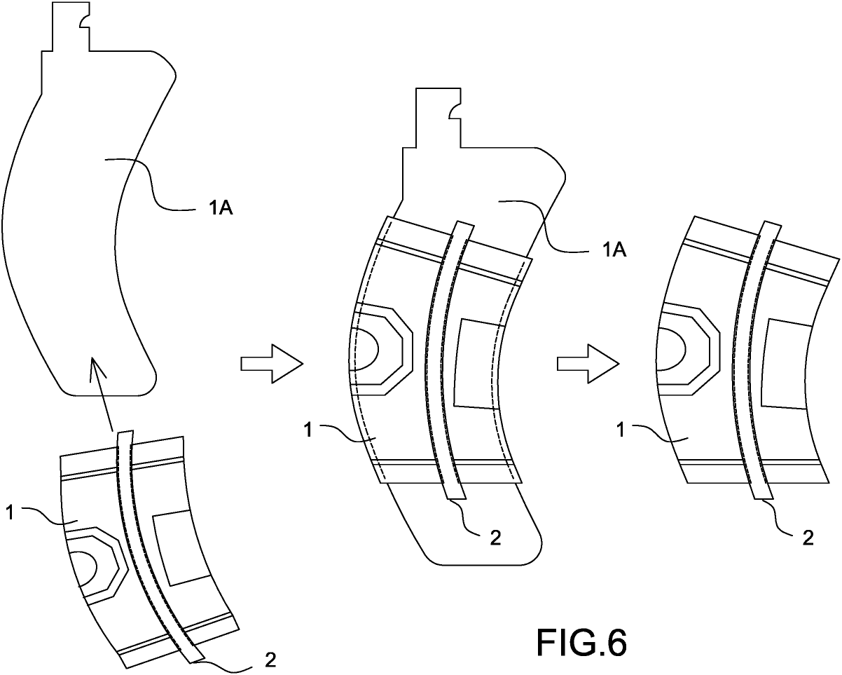

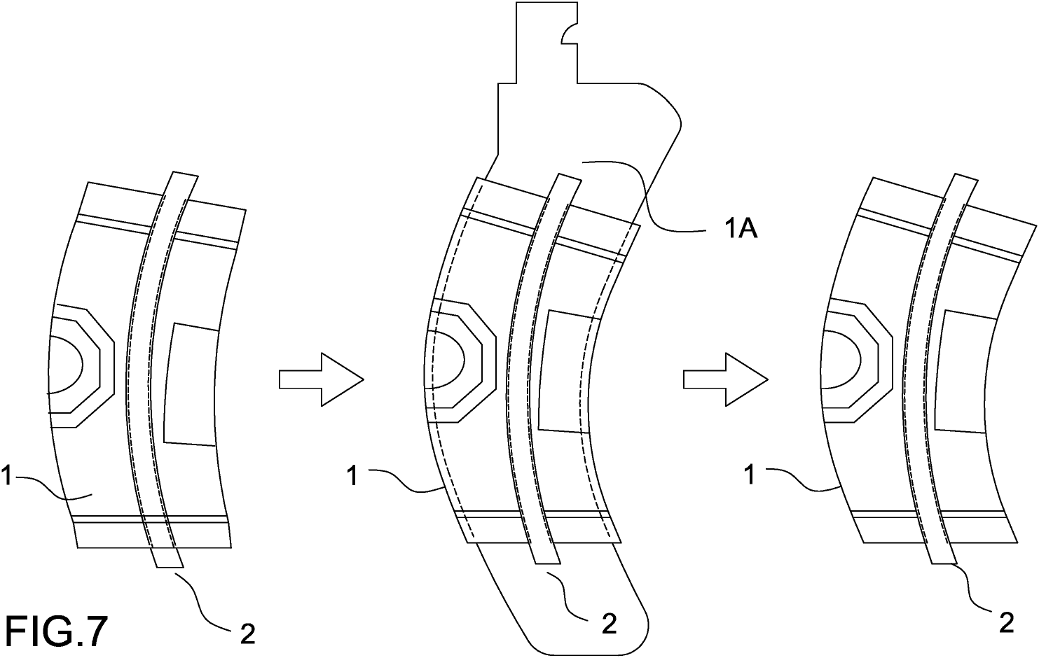

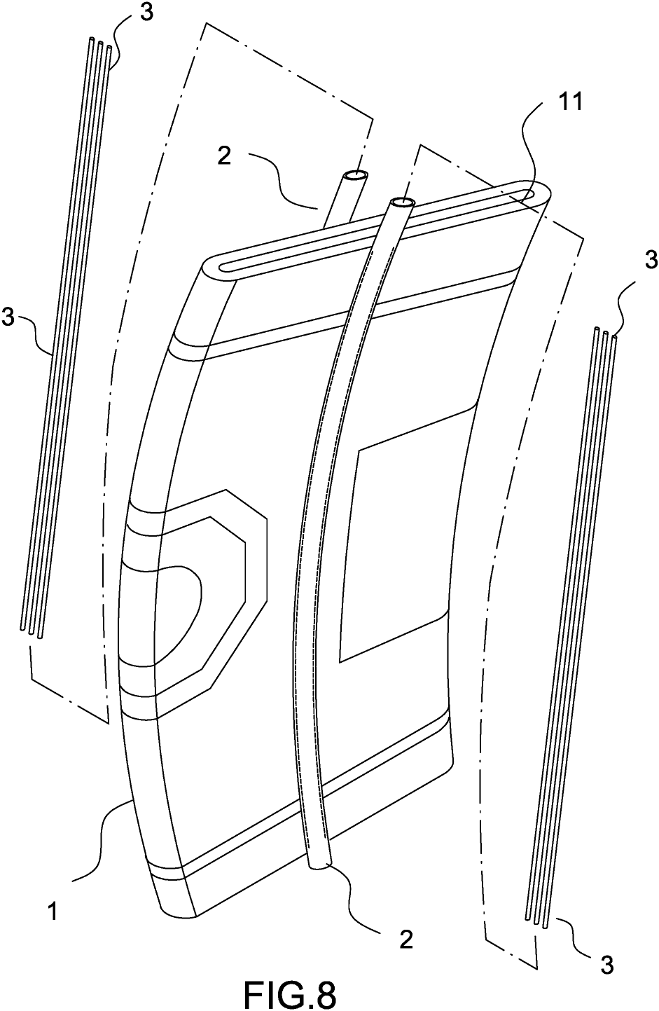

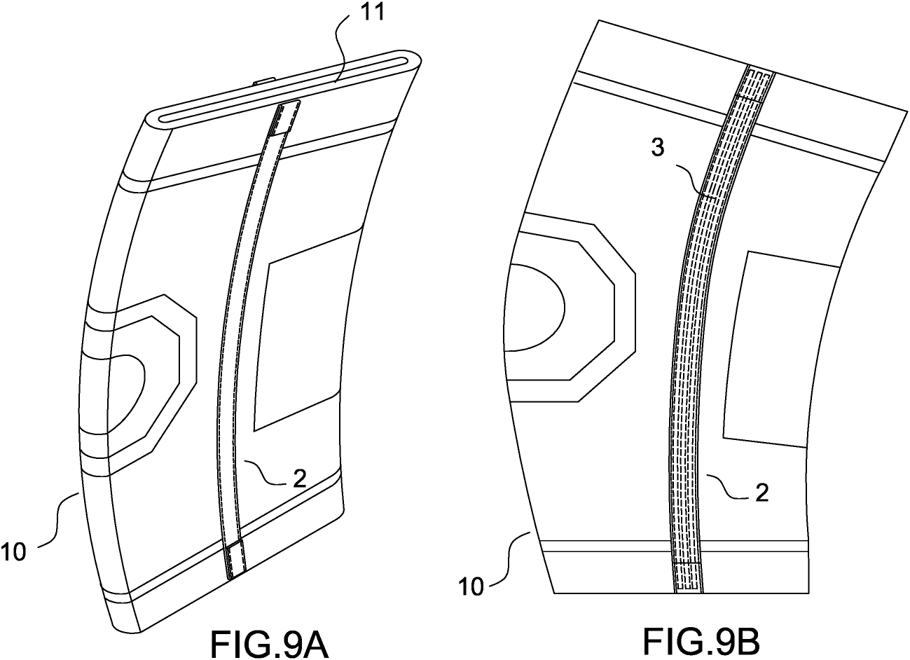

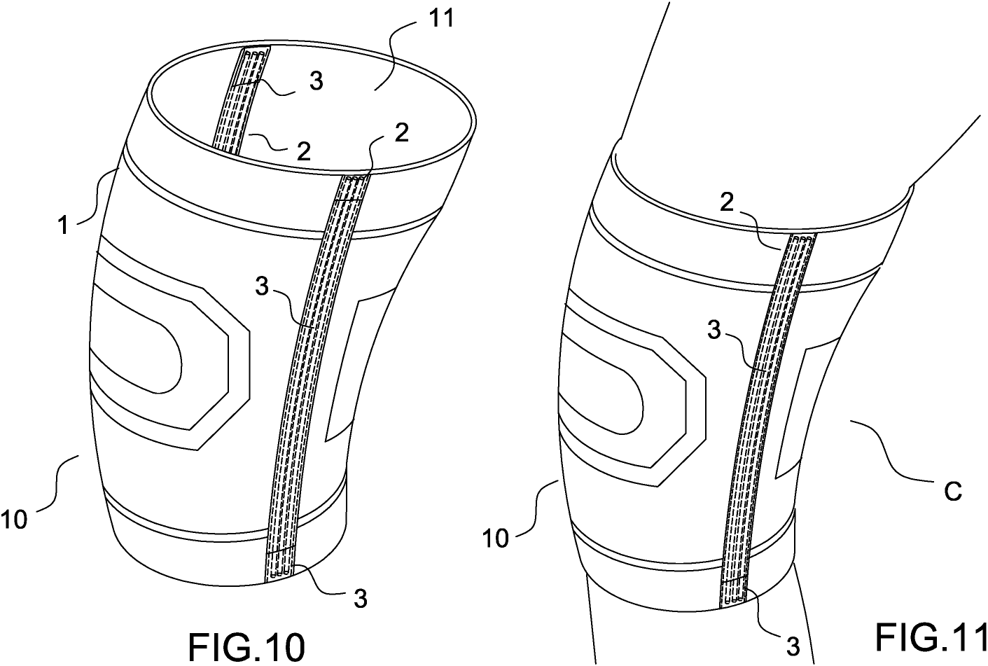

Referring to FIGS. 3 to 10, a process of making a knee brace 10 in accordance with the invention comprises the steps of putting a tubular cushioning body 1 on a flat mold 5 for shaping (see FIG. 3), activating a sawing device 6 to secure a fabric 2A to either side of the cushioning body 1 aligned with a hollow portion of the mold 5 to form two flexible sheaths 2 (see FIGS. 4 and 5), removing the mold 5 out of the cushioning body 1, putting the cushioning body 1 on a bent, flat mold 1A for shaping, and removing the mold 1A out of the cushioning body 1 (see FIG. 6) or alternatively inserting the mold 1A into the cushioning body 1 and removing the mold 1A out of the cushioning body 1 (see FIG. 7), and inserting a plurality of elastic strips 3 into each of the sheaths 2 to form a knee brace 10 having a hollow 11 (see FIGS. 8, 9A and 9B). The hollow 11 can be enlarged by inserting the hand thereinto (see FIG. 10). Further, the number of the elastic strips 3 can be increased or decreased as desired. A sufficient strength and flexibility of the knee brace 10 is provided by the elastic strips 3. Thus, a person wearing the knee brace 10 on his or her body parts may feel a degree of comfort without worrying it being loosened. Further, the life expectancy of the knee brace 10 is prolonged.

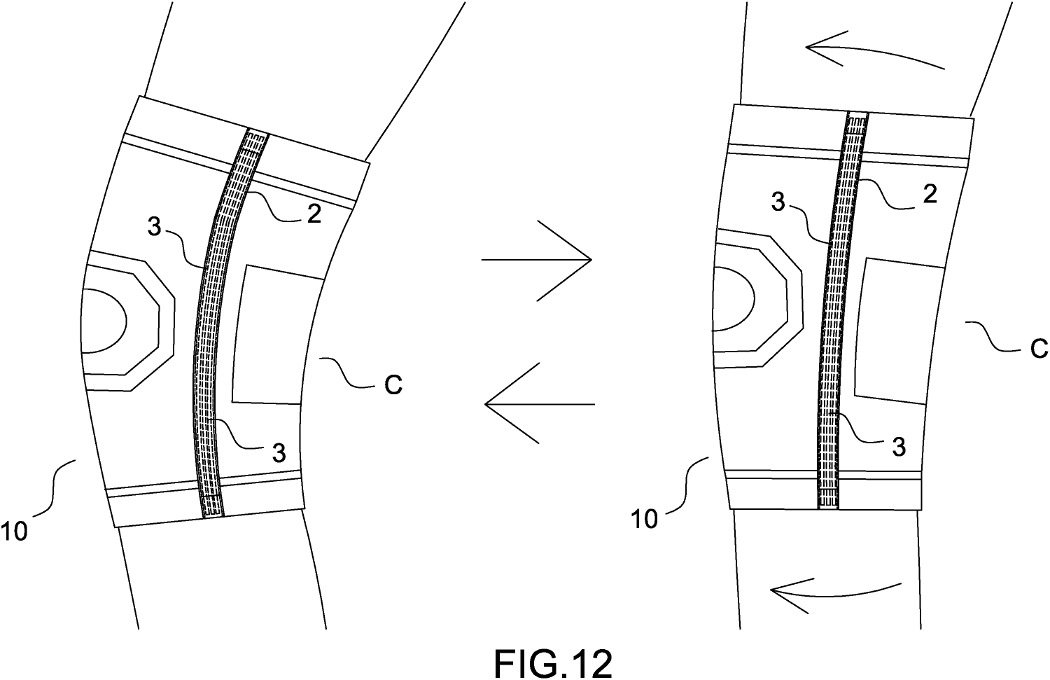

Referring to FIGS. 11 to 12, the knee brace 10 can be put on the knee C of a person in a first configuration of the invention. The leg may be straightened by pivoting about the knee C as indicated by two solid arrows after bending in exercise. Advantageously, the elastic strips 3 do not exert a force to oppose the strengthening force (or bending force in a bending motion).

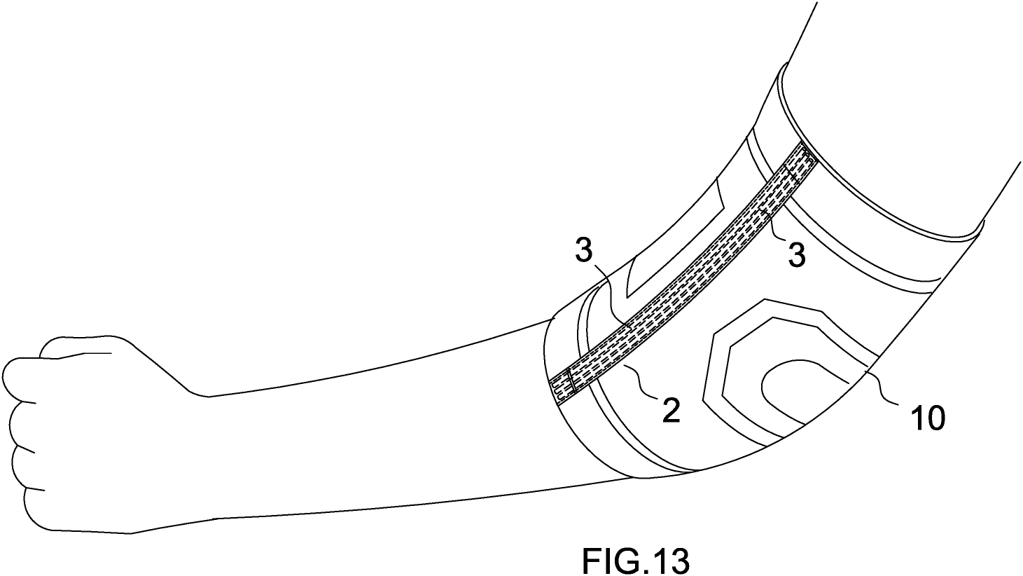

Referring to FIG. 13, the brace 10 can be put on the elbow of the person in a second configuration of the invention. The hand may be straightened by pivoting about the elbow after bending in exercise. Advantageously, the elastic strips 3 do not exert a force to oppose the strengthening force (or bending force in a bending motion).

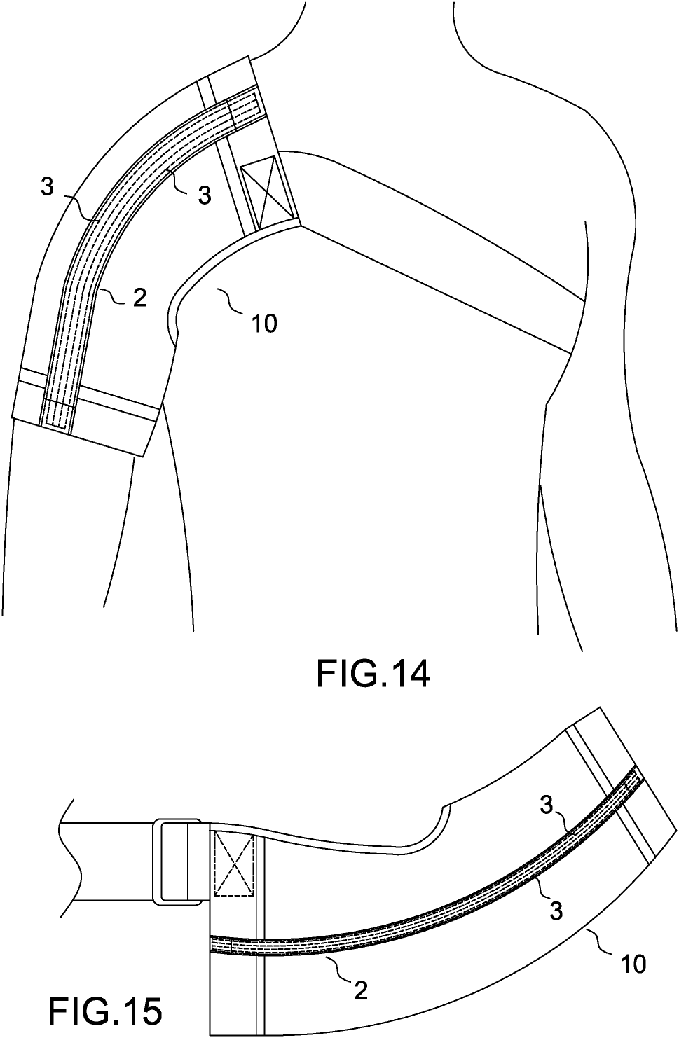

Referring to FIGS. 14 and 15, the brace 10 can be put on a joining portion of the hand and the shoulder of the person by putting a looped strap (not labeled) over the shoulder in a third configuration of the invention. The hand may be straightened to align with the shoulder by pivoting about the joining portion after bending in exercise. Advantageously, the elastic strips 3 do not exert a force to oppose the strengthening force (or bending force in a bending motion).

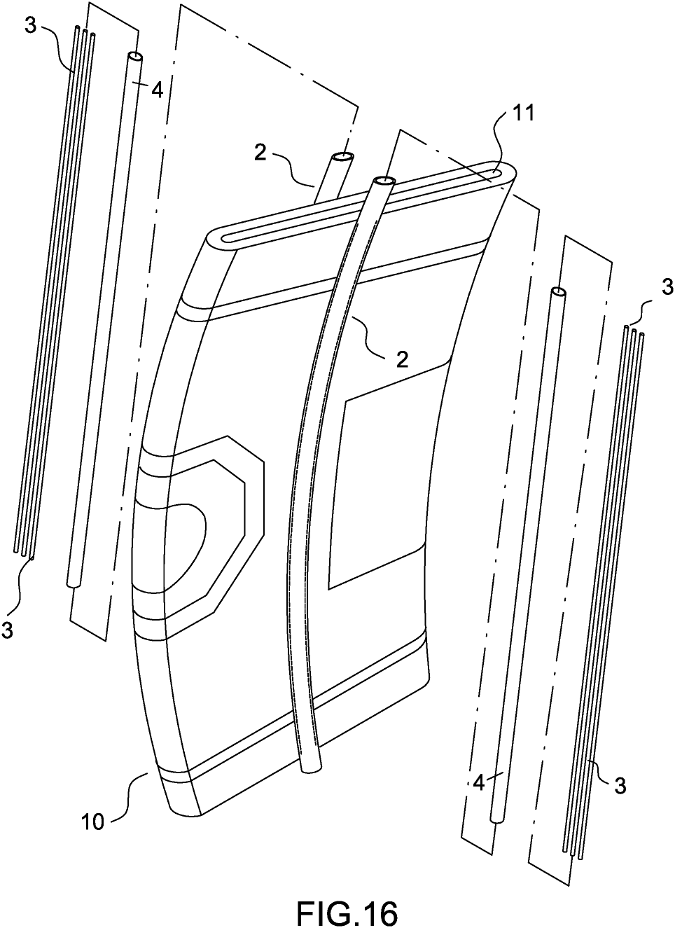

Referring to FIG. 16, alternatively, the elastic strips 3 are inserted into each of two tubes 4 which are inserted into the sheaths 2 respectively in another embodiment of the invention.

While the invention has been described in terms of preferred embodiments, those skilled in the art will recognize that the invention can be practiced with modifications within the spirit and scope of the appended claims.

What is claimed is:

1. A knee brace, comprising:
a tubular cushioning body;
two sheaths disposed at two opposite sides of the tubular cushioning body respectively; and
two sets of a plurality of elastic strips inserted into the sheaths respectively.

2. The knee brace of claim 1, further comprising two tubes with the sets of the plurality of elastic strips inserted thereinto respectively, wherein the tubes are inserted into the sheaths respectively.
